(19) United States
(12) Patent Application Publication
(10) Pub. No.: US 2025/0005831 A1
(43) Pub. Date: Jan. 2, 2025

(54) SYSTEM AND METHOD FOR CREATING SMILE MOCKUPS AND FACILITATING ASSOCIATED DENTAL PROCEDURES

(71) Applicants: Billie Prisby, Boca Raton, FL (US); Sham Chotai, Redwood City, CA (US); Daniel Roviros, Coral Springs, FL (US)

(72) Inventors: Billie Prisby, Boca Raton, FL (US); Sham Chotai, Redwood City, CA (US); Daniel Roviros, Coral Springs, FL (US)

(21) Appl. No.: 18/756,843

(22) Filed: Jun. 27, 2024

Related U.S. Application Data
(60) Provisional application No. 63/510,393, filed on Jun. 27, 2023.

(51) Int. Cl. G06T 11/60; G16H 30/20

(52) U.S. Cl. CPC G06T 11/60; G16H 30/20; G06T 2200/24; G06T 2210/41

(57) ABSTRACT

A method includes capturing at least one image of a face of a user. The method includes performing preprocessing on at least one image. The method includes receiving a selection of one smile template by the user. The method includes applying the at least one image and the selection to one or more machine learning algorithms. The one or machine learning algorithms are trained to generate one or more modified images that transform a smile based on the selection of one smile template and trained to output one or more procedures required to transform the smile. The method includes transmitting the one or more modified images and the one or more procedures to the user.

### CROSS-REFERENCE TO RELATED APPLICATIONS

This application claims the benefit of priority of U.S. provisional application No. 63/510,393, filed Jun. 27, 2023, titled “SYSTEM AND METHOD FOR CREATING SMILE MOCKUPS AND FACILITATING ASSOCIATED DENTAL PROCEDURES” the entire contents of which are herein incorporated by reference.

### FIELD

The present disclosure relates to image generation and editing and, more particularly, to artificial intelligence-enhanced dental image generation and editing.

### BACKGROUND

Cosmetic dentistry often requires software tools to plan and visualize potential dental procedures. Traditional dental applications are designed for desktops and take time to manipulate manually, like image editing programs. For example, traditional dental applications take at least 10 minutes to produce one conversation-starting image that is printed. The traditional dental applications lack guardrails and automation to ensure you do not need to be a dentist to use the system. In addition, there is no training, education, or consultative features.

As can be seen, there is a need for systems and methods that address the above drawbacks.

### SUMMARY

In one aspect of the present disclosure, a method includes capturing at least one image of a face of a user. The method includes performing preprocessing on at least one image. The method includes receiving a selection of one smile template by the user. The method includes applying the at least one image and the selection to one or more machine learning algorithms. The one or machine learning algorithms are trained to generate one or more modified images that transform a smile based on the selection of one smile template and trained to output one or more procedures required to transform the smile. The method includes transmitting the one or more modified images and the one or more procedures to the user.

### BRIEF DESCRIPTION OF THE DRAWINGS

FIGS. 1-20 are diagrams of graphical user interfaces of a dental image system and application, according to aspects of the present disclosure;

FIG. 21 is a diagram of a dental imaging system, according to aspects of the present disclosure;

FIG. 22 is another diagram of the dental imaging system, according to aspects of the present disclosure; and

FIGS. 23A-23C are flow diagrams illustrating dental imaging processes, according to aspects of the present disclosure.

### DETAILED DESCRIPTION OF THE DISCLOSURE

The following detailed description is of the best currently contemplated modes of carrying out exemplary embodiments of the disclosure. The description is not to be taken in a limiting sense but is made merely for the purpose of illustrating the general principles of the disclosure, since the scope of the disclosure is best defined by the appended claims.

Broadly, an embodiment of the present disclosure provides a dental imaging system and method for dental examinations and diagnosis and, more particularly, to information technology, image data processing, dental procedures, and scheduling. The dental imaging system operates to create smile mockups. The system uses facial and dental features from captured images generated by a user (or prospective patient), 3D image processing technology, and a robust library of desirable smiles to create an enhanced smile mockup. Facial features of captured images focus on soft and hard tissues that contribute to a user's smile. Dental features focus on a user's teeth and their relation to the soft tissue in the user's oral cavity, such as the gums. The dental imaging system and method substitute user facial and dental features with one or more smile enhancement and face enhancement features using one or more user selections from the library of desirable smiles.

The dental imaging system generates one or more smile profiles for review and evaluation by a prospective patient, dental assistant, dental practitioner, or any combination thereof and makes planning and scheduling a dental procedure or surgery easier and more efficient. More specifically, the system uses federated and sponsored systems to reduce the amount of time and resources needed to plan a procedure, improve quality of care through greater choices, and reduce redundant network services and over utilization of system resources.

The dental imaging system operates as a conversation-starting tool for clinical teams and patients with AI and code that is building specialized AI and using AI for video that talks about and shows patients smile possibilities in under 60 seconds. Once generated, dental mockups are then texted to the said patients device instantly. made for dental team members, not doctors. As such, dental team members can use the dental mockup to start conversations and become more strategic clinicians.

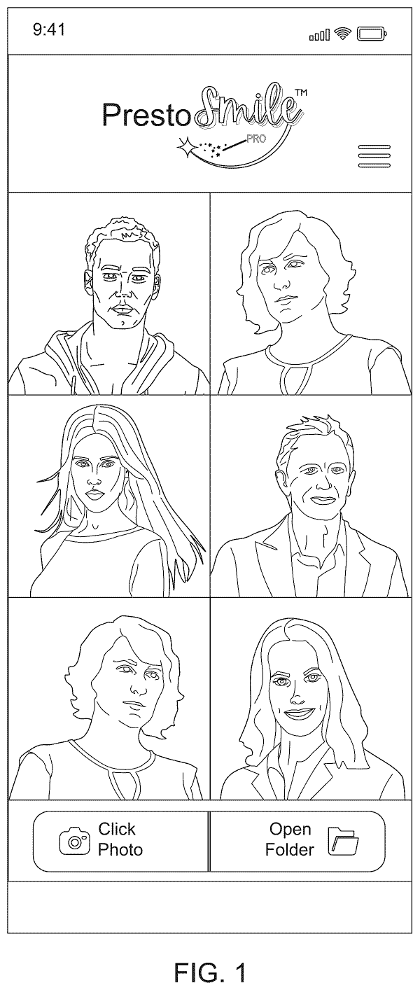

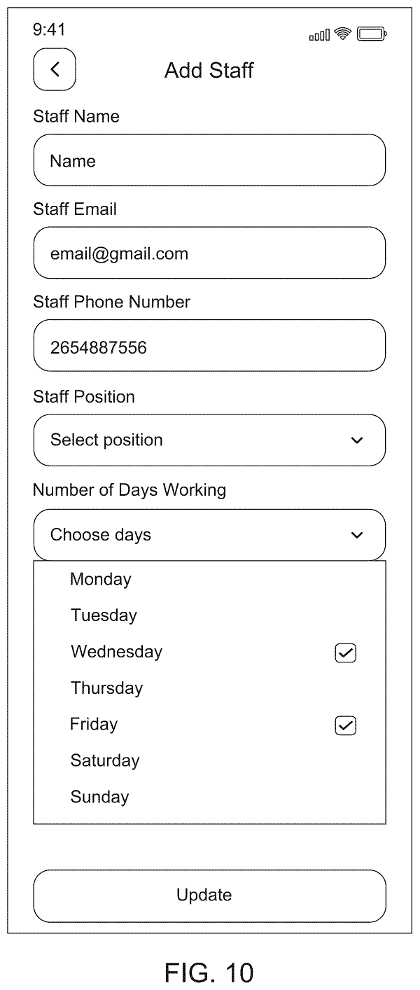

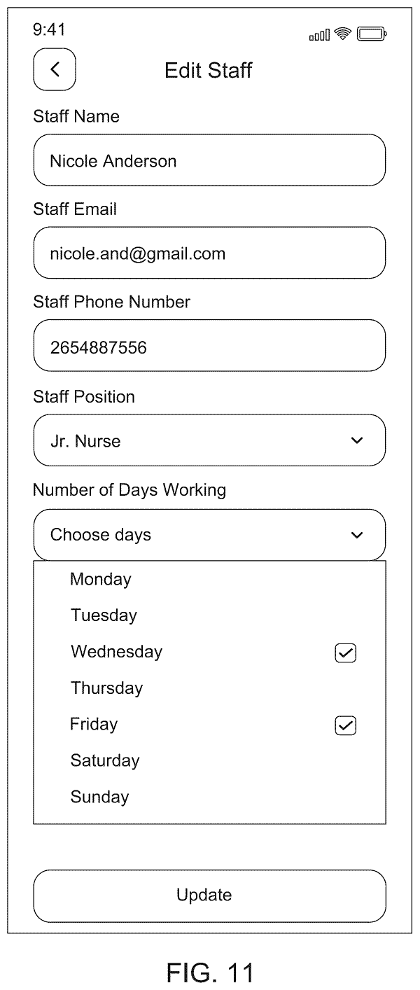

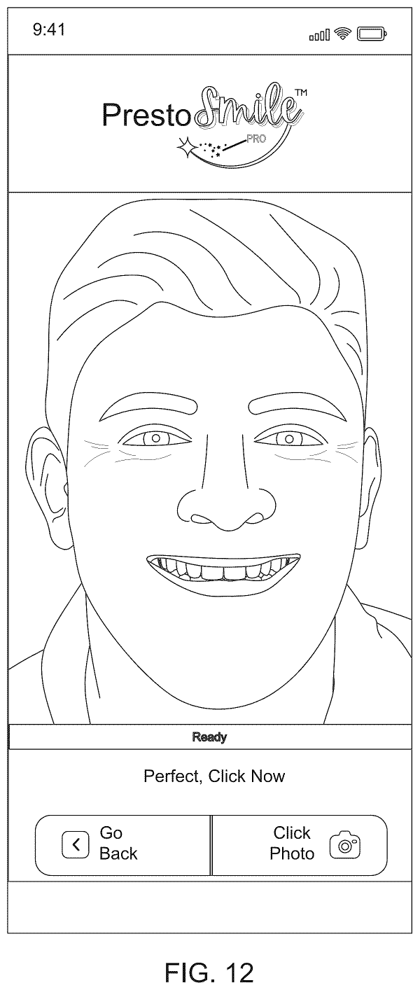

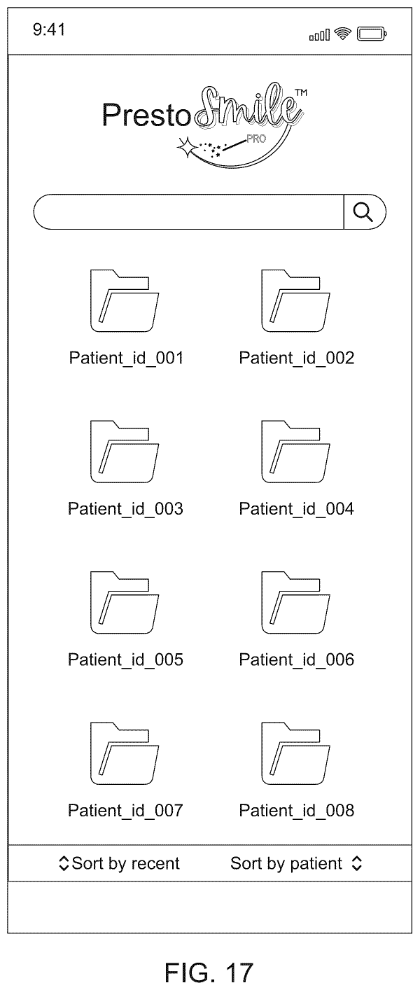

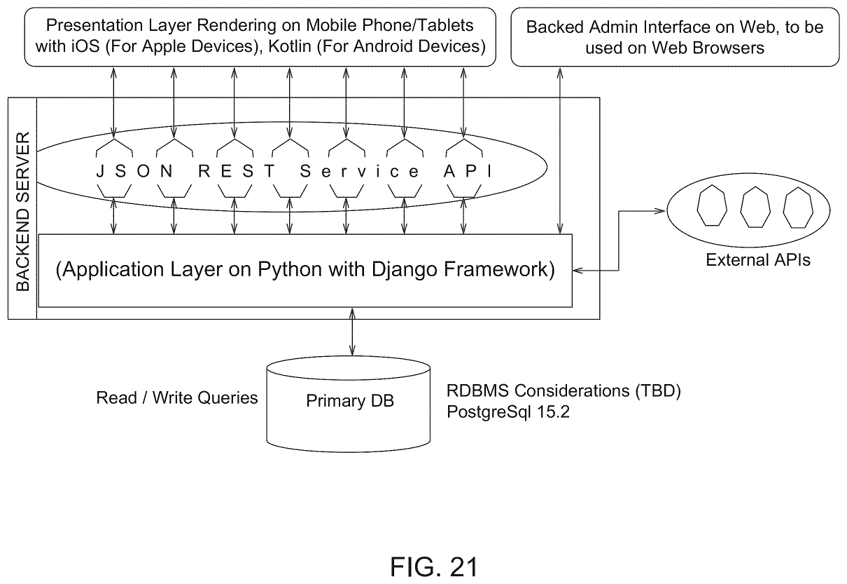

Referring now to FIGS. 1-21, FIG. 21 illustrates a dental imaging system, according to aspects of the present disclosure. While FIG. 21 illustrates examples of components of dental imaging system, additional components can be added and existing components can be removed and/or modified.

As illustrated in FIG. 21, the dental imaging system includes one or more computer devices, e.g., backend server, that provides one or more application services for generating dental images. For example, the backend server can implement an application layer that provides one or more application programming interfaces (APIs), e.g., Javascript Object Notation (JSON) REST API, that allows one or more applications operating on one or more user devices to communicate with the backend server. One or more applications operating on the user devices provide a presentation layer for delivering one or more graphical user interfaces (GUIs) to users. The user devices can include one or more electronic devices such as a laptop computer, a desktop computer, a tablet computer, a smartphone, a thin client, and the like. The user devices can communicate with the backend server via one or more networks.

In embodiments, the dental imaging system (also referred herein as Presto Smile) is configured to capture one or more images of a patient and/or user and generate one or more smile mock-ups that represent prospective dental work that can be performed on prospective patients. The dental imaging system is an innovative dental practice management system powered by specialized artificial intelligence (AI) technology, e.g., one or more machine learning algorithms, designed to revolutionize the patient experience and streamline workflows in the dental industry. The dental imaging system includes a video AI consultative tool that educates patients and showcases smile possibilities using advanced AI algorithms. The dental imaging system provides an intuitive dashboard, patient management capabilities, AI-driven smile simulations, staff management tools, and customer support functionalities. The dental imaging system is a cutting-edge dental conversation system that leverages AI technology to enhance patient engagement, optimize workflow efficiency, and improve overall practice performance.

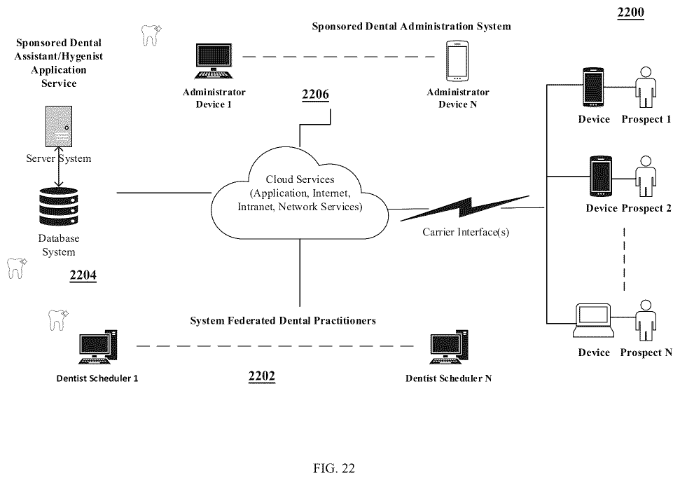

For example, FIG. 22 illustrates a dental imaging system 2200, that includes dental practitioners-federated system 2202, sponsored dental support staff application service 2204, sponsored dental administration system 2206, and devices for prospective patients 1-N, according to an example embodiment. Dental practitioners-federated system 2202 comprises dentist schedulers 1-N for sending, receiving, processing, or any combination thereof prospective patient and dentist records data, such as profile data, calendar data, smile profile data, and edits or updates thereto.

Sponsored dental assistant application service 2204 comprises a server system and database system for sending, receiving, creating, processing, or any combination thereof algorithmic models, datasets, tagged datasets, prospective patient records data, dentist records data, or any combination thereof.

Sponsored dental administration system 2206 comprises administrator devices 1-N for sending, receiving, creating, processing, or any combination thereof statistical algorithms, algorithmic models, datasets, dataset tags, federated rules, processes, standards, practices, or any combination thereof, and other system, e.g., Unix or Unix-like, administration functions, such as database administration, system access, authorization, and authentication, and security (PKI, e.g.).

Devices for prospects 1-N comprise client software for sending, receiving, creating, processing, or any combination thereof prospective patient records data, dentist records data, or edits and updates thereto. Records data can include profile data, calendar data, smile profile data, or edits and updates thereto. Devices for prospects 1-N, sponsored dental administration system 2206, sponsored dental assistant application service 2204, and federated system of dental practitioners 2202 can send, receive, process, create, generate, or any combination thereof data using federated services, industry standard services, open-source standards, or any combination thereof.

In an embodiment, system 2200 can be implemented as part of a set of cloud-based services, e.g., application, Internet, intranet, network services, or any combination thereof, provided by a third party. Devices for prospective patients 1-N can be communicably coupled to the set of cloud-based services through a system of network and gateway devices and one or more carrier interface(s).

As used herein, user, prospect, patient, dentist, and assistant refers to data structure(s) that comprise medical, dental, insurance, personal, professional, or other data needed to form a system account and service account. Celebrity smiles image data refers to data structures that comprise facial and smile imaging data for various celebrities or famous people. Prospect-patient image data refers to data structures that comprise facial and smile imaging data of prospective patients and patients. Smile profile data refers to data structure(s) that comprise prospective patient facial and teeth imaging data. Enhanced smile profile data refers to enhanced prospective patient facial and teeth imaging data. Enhanced refers to the result of conflating or substituting a celebrity smile with a prospective patient smile. Image evaluation and “create facial and dental constructs from image evaluation results” refers to 3D imaging and statistics-based modeling processes that identify objects in prospective patient facial and teeth image data, compares the identified objects against one or more celebrity smiles image data, and adjusts parametric values of the identified objects accordingly for presentation. The enhanced smile(s) profile data can be stored, presented to the prospective patient, sent to one or more referrals, or any combination thereof. The one or more referrals can refer to dentist(s), assistant(s), or both. Referral notification messages can comprise dentist(s), assistant(s), or both, scheduling information, and prospective patient profile information. Referral notification messages can be sent to prospective patients, dentists, assistants, or any combination thereof. Statistics-based modeling refers to the use of a trained algorithmic model that has been trained using a statistics-based algorithm and celebrity smiles dataset. The trained algorithm model comprises a parameter space configured for transposing parametric values based on evaluation of image objects. Start (instantiate) refers to heap based data structures and variables. End refers to respect algorithm completion after initiation and execution.

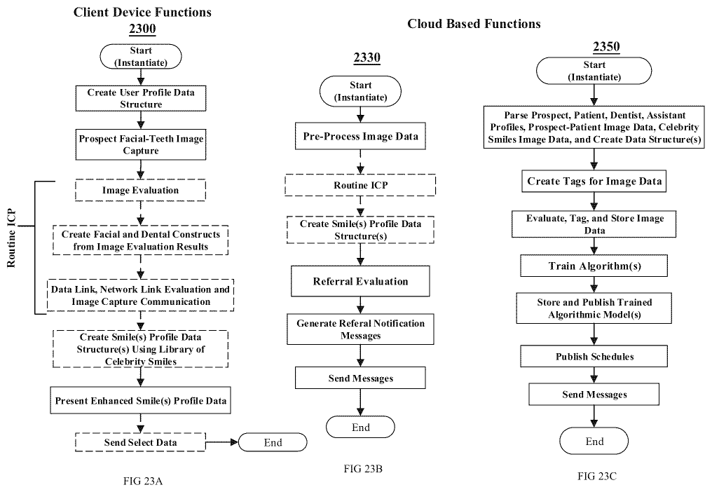

In an exemplary process, as illustrated in FIGS. 23A, 23B, and 23C, a prospective patient can initiate an application on a user device that communicates with the dental imaging system. Then, the prospective patient can create a user profile, which is stored a user profile data structure in the dental imaging system. Next, one or more prospect facial-teeth images can be captured. The images can be captured by the user device and/or one more imaging devices of the dental practitioners. The images are then transmitted to the dental imaging system for image capture processing (ICP). In ICP routine, the dental imaging system performs image evaluation and creates facial and dental constructs from the image evaluation results. In embodiments, the dental imaging system applies one or more machine learning algorithms and a smile library to generate a smile mock-up. The dental imaging system can create smile profile data using the smile library. The smile mock-ups can be presented to the prospective patient using, for example, the user device.

In embodiments, the smile library is a collection of pre-trained smiles generated from the dental imaging system foundational model and AI to tailor a smile to a specific patient's facial structure, geometry, and soft tissue. It is an optimization process to select potential smiles that are suitable/appropriate for a patient's specific facial structure and based on established cosmetic dental practice and art. The library can include celebrity smiles and additional smile styles.

The dental imaging system also includes an AI Assistant “BillieAI” or “sidekick”. BillieAI is a virtual assistant that helps the dental assistant or hygienist sell cosmetic dentistry and is integrated with all of Presto Smiles capabilities. It's a process for having a three way conversation with the patient to explain dental treatments options based on the patients demographic and personality profile. The trained AI model reasons completions based on prompts provided through an audio conversation with BillieAI. The capability is based on industry standards of care and addresses concerns and apprehensions patients may have about a treatment plan. The sidekick relieves the assistant of explaining treatments and provides a standardized way of selling treatments to patients.

In embodiments, the dental imaging system can utilize one or more machine learning algorithms to generate the smile mock-ups. The one or more machine learning algorithms can be trained using patient data. In the training process, the dental imaging system can parse prospect, patient, dentist, assistant profiles, prospect-patient image data, celebrity smiles image data, and create data Structure(s). Then, the dental imaging system can create tags for the image data and can evaluate, tag, and store the image data. The tagged image data can be used to train the one or more machine learning algorithms. Once trained, the algorithmic models and be stored and published.

In embodiments, the dental imaging system takes a 2D digital image of a patient and transforms it into a 3D model of the facial and teeth structures. Specifically, the model captures information about the patient's facial features and texture, tooth contours, geometry, and soft tissues. The system then leverages a pre-trained foundational model of smiles to learn and generate a smile that is optimized for the patient's facial features and preferences. Specifically, the dental image generation system enhances the 3D model to render 2D facial images so that the patient can select a smile that is aesthetically pleasing. The image generation system uses deep learning architectures such as generative adversarial networks (GAN) and Diffusion models that operate on pre-trained clinical 3D models of teeth and facial structures and generate possible smiles. The deep learning architecture also uses a novel crontraint model that categorizes patients and guides deep learning through negative feedback mechanisms to produce smiles that are appropriate for each patient.

Once the one or more machine learning algorithms are trained, the captured images of the prospective patient can be applied. The image data can be pre-processed. Then, the ICP routine can be performed to create the smile profile data structure. The mock-ups can be then used by the dental professionals in a referral evaluation and to generate referral evaluation messages.

Below our various specific facial and dental features that can be used by the dental imaging system to create a smile mock-up.

Facial composition—the dental imaging system can take into consideration facial symmetry, alignment, and proportions to design a smile and works with a team of specialists. The dental imaging system can use prior designs from orthodontists, orthognathic surgeons, periodontists, and plastic surgeons to generate the smile mock-up.

Inter-pupillary line—This is an imaginary line drawn between the centers of the eyes' pupils. It should be parallel to the occlusal plane, an imaginary surface that touches the biting edges of the incisors (the four front teeth) and the biting surface of the back teeth.

Lips—Lips play a major role in smile design because they border the teeth. The shape, movement, and flexibility of the lips can determine a smile's appearance. Gummy smile can also be considered.

Lip length—the dental imaging system can measure from the point below the nose to the lower border of the upper lip in its resting position. The upper lip tends to increase with aging, so decreasing this distance, or at least the appearance of this distance could help a patient look younger.

Lip mobility—This measures the distance between the top lip in the resting position to when it's in the high smile position. Some patients have hypermobility, which causes a gummy smile.

Dimensions—The face can be divided into two dimensions: horizontal and vertical. The components of the horizontal dimension include: smiles design—The contact area is another design factor; Face width: Should be five times the width of the eye; Nose width: Should equal the inner intercanthal distance, or the distance between the inner corners of the eyelids; and Mouth width: Should equal the interpupillary distance, the distance between the centers of the pupils of the eyes.

The vertical dimension can be divided into three equal parts: Upper third: The lowest point of hairline between the eyebrows to the eyebrow line; Middle third: The eyebrow line to the base of the nose; and Lower third: The base of the nose to the base of the chin.

Face shape—There are six facial shapes that you can observe by looking at the front of the face—heart, long, oval, triangle, square, and round. Looking at the face in profile, you can observe one of three profile types—convex, straight, and concave.

Dental composition Teeth—smile design zenith points—A smile design is based on factors like zenith points. Dental midline (line between two upper and two lower front teeth) and Incisal length (length of four front teeth).

Tooth dimensions—Zenith points (the highest points of the gumline arch on teeth); Axial inclination (the angle formed between the long axis of the tooth and the bone it comes from); Interproximal contact area (ICA; the area where two neighboring teeth make contact); Interproximal contact point (IPA; the contact area between two teeth); Incisal embrasure (the space between two endings of adjacent front teeth)

Other factors: Gender, personality, and age; Symmetry and balance (smile designing—Smile line is at the bottom edge of your top teeth); Soft tissue; Gingival health; Gingival line; Interdental embrasure (space between adjacent teeth); Smile line; Dental golden ratio; and dental golden proportion (When viewed head-on, the width of each front tooth beginning in the center and going outwards is 60% of the width of the adjacent tooth, with a ratio of 1.6:1:0.6. Because patients have varying arch forms, lip anatomy, and facial proportions, it's impossible to exactly reproduce the golden ratio rule, but dentists will use it as a loose guide.

Once the mock-up is generated, the patient and dental professionals can preview the proposed design. As such, the patient can see the smile preview before they begin the procedure. They can work with the dentist to modify color, shape, and length to create their ideal smile. This helps make the final result more accurate, as it is done with an oral scanner and 3D printer.

The dental imaging system can also be used to find a dentist near them, work out payment plans, and schedule their appointments. Additionally, the patient can continually use the dental imaging system to book appointments directly with the new dentist and the dental offices can be able to follow up via the dental imaging system with their patients with appointments, payment status and treatment needed or wanted.

The dental imaging system can also be access remotely to generate the mock-up. For example, the patients can perform the evaluation in the privacy of their home where they can save embarrassment if they don't like their smile and where they can mockup their smile, get an approved payment plan of time and get an appointment with a qualified doctor.

In embodiments, the dental imaging system can generate one or more GUIs to perform the methods and processes described above. FIGS. 1-20 illustrates examples of the GUIs that can be used by a user. FIG. 1 illustrates the user-friendly dashboard interface, where clinicians can seamlessly capture patient images and utilize AI algorithms to demonstrate potential smile transformations to patients. The system's innovative video AI consultative tool, coined AI Billie, provides personalized consultations and guides users through various functionalities.

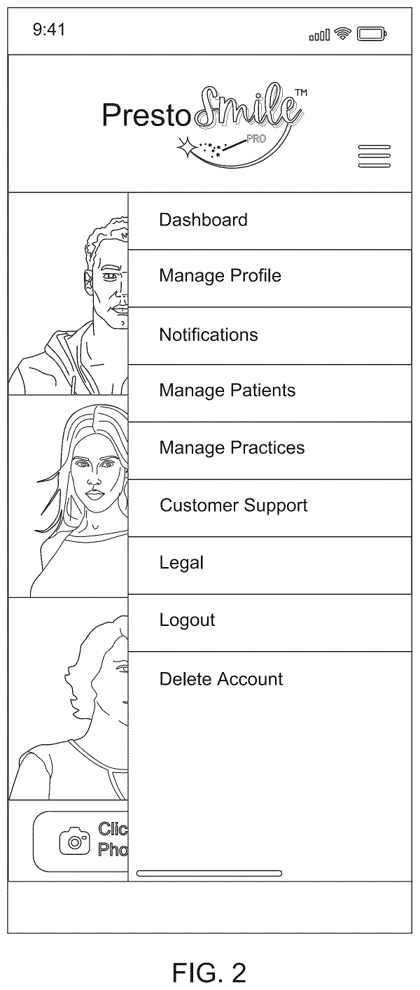

FIG. 2 showcases the hamburger menu, granting dental team members access to essential features such as the dashboard, profile settings, notifications, and interactive video conversations with AI Billie. Additionally, the menu includes options to manage patient charts, dental practices, team members, customer support inquiries, legal information, account management, and logout functionalities.

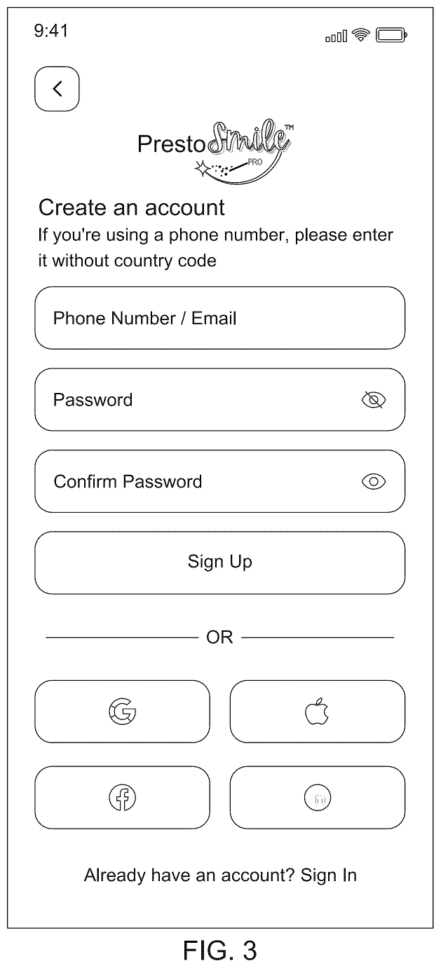

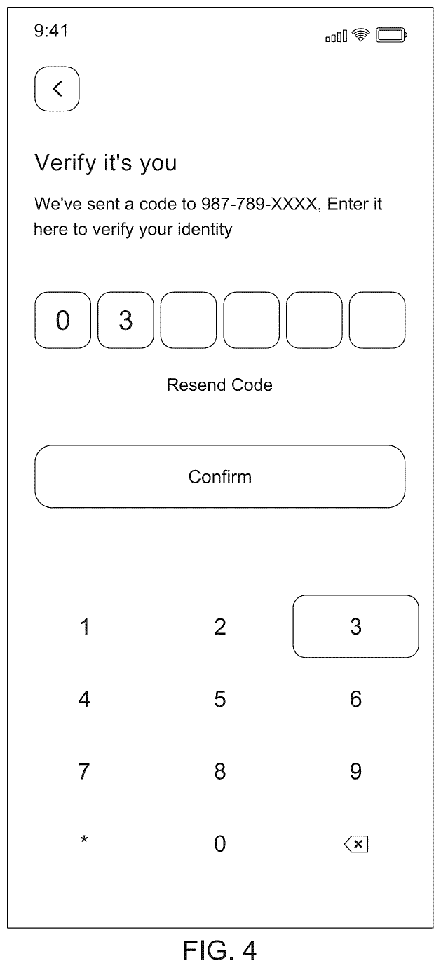

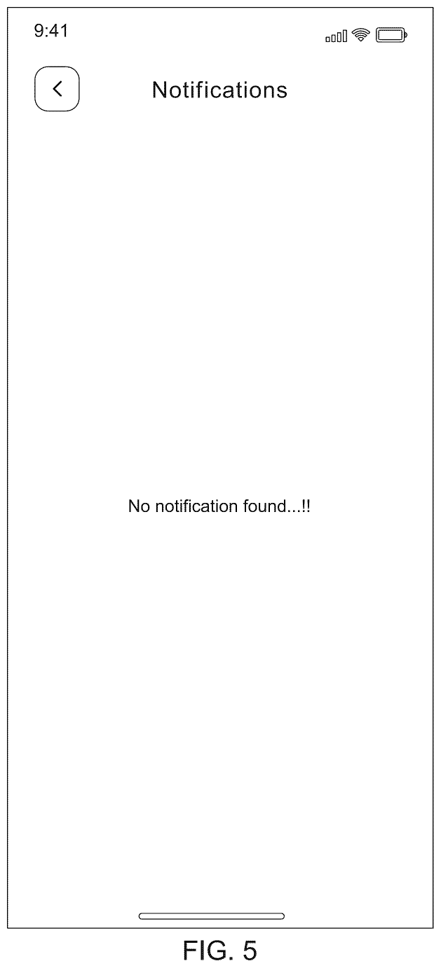

For new users, FIG. 3 depicts a step-by-step guide for dental practices to create an account within the dental imaging system. Upon registration, FIG. 4 demonstrates the use of a one-time password (OTP) for account verification, ensuring data security and user authentication. The system also provides notifications (FIG. 5) to deliver training videos for team members to enhance their skills or acknowledge their achievements, thereby promoting continuous professional development within the practice.

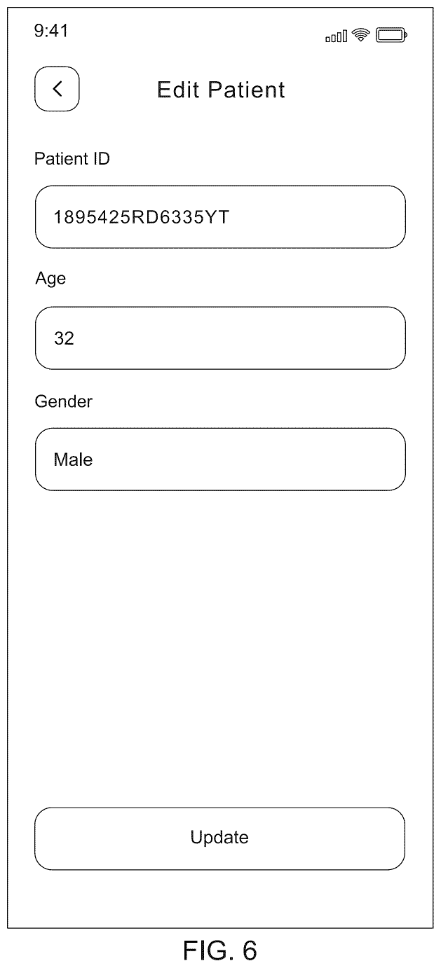

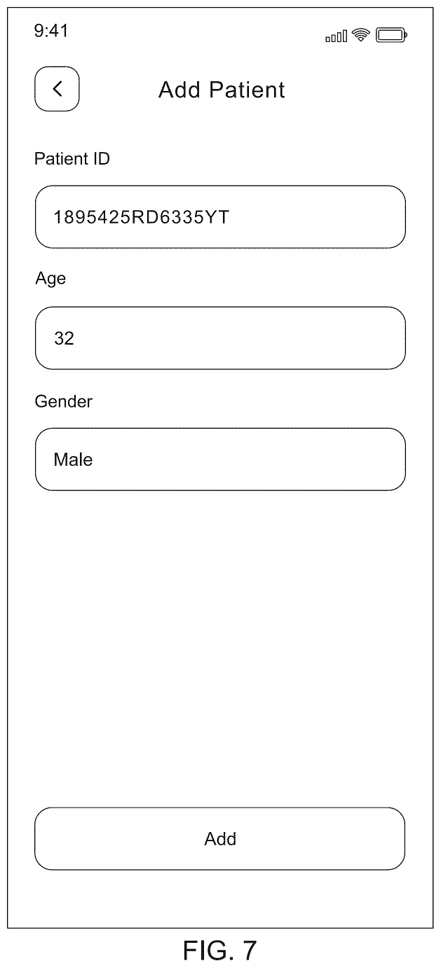

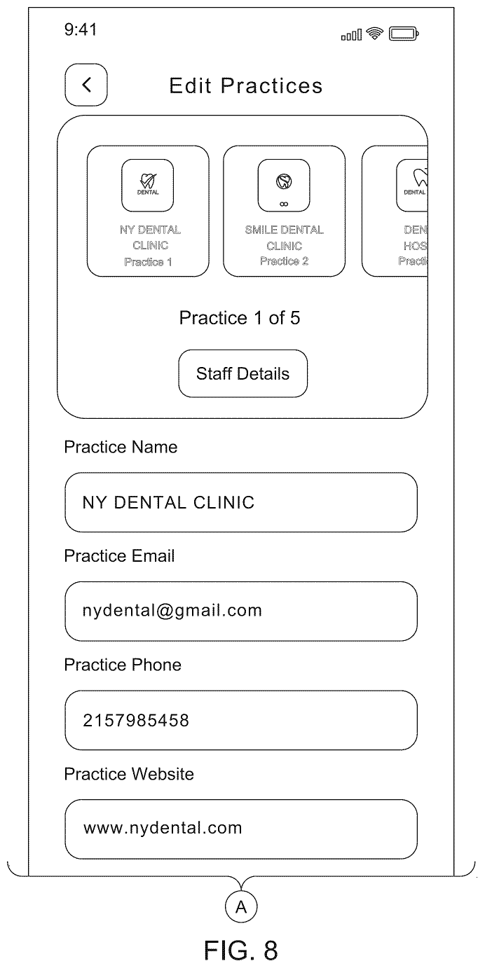

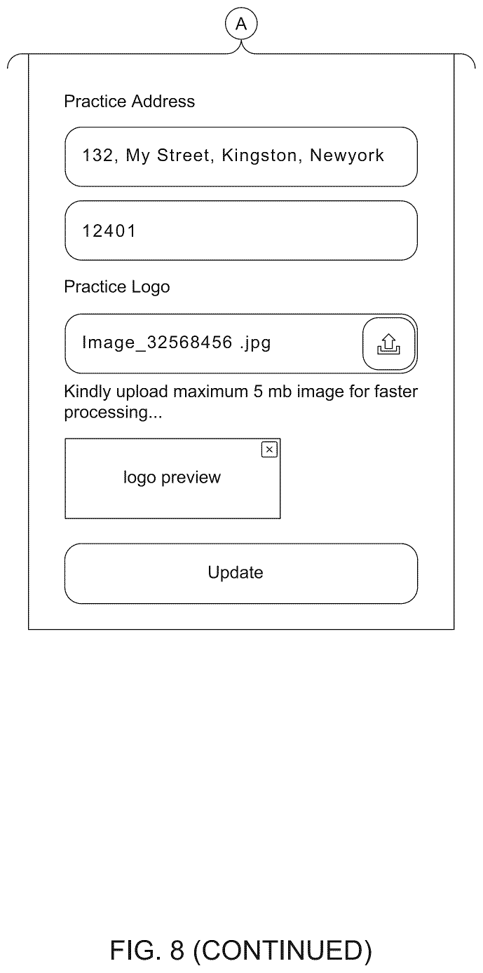

To facilitate patient management, FIGS. 6 and 7 enable users to edit existing patient records or add new patients effortlessly. Additionally, FIG. 8 allows for the seamless addition or modification of practice information, including practice details and staff profiles. Users can upload staff information, such as email addresses, phone numbers, positions, and work schedules, to streamline practice operations and communication.

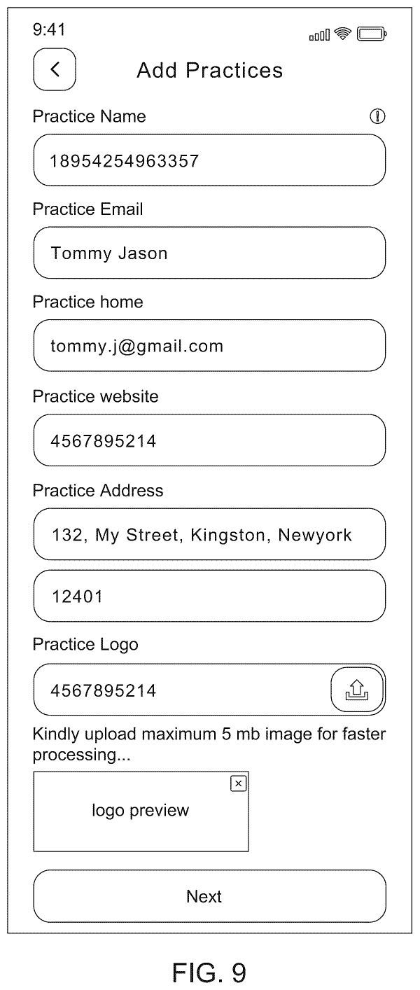

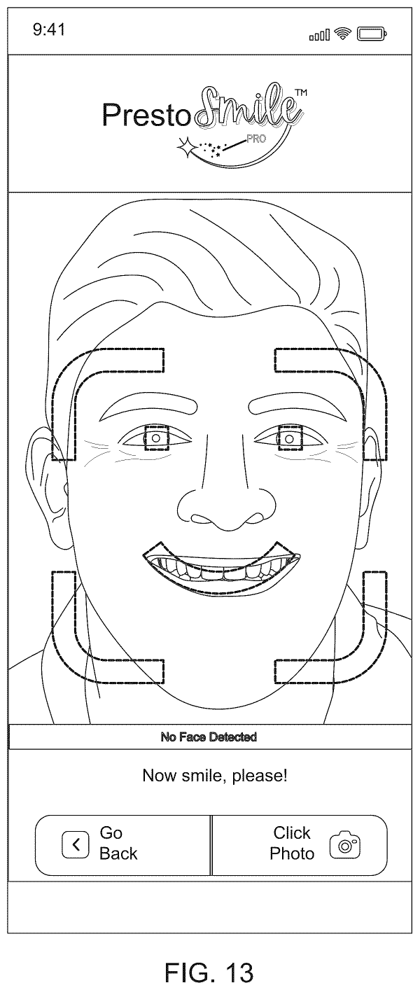

FIG. 9 showcases the integration of a logo feature that enables users to instantly send branded smile mockups to patients via email or mobile devices, as well as sync this information with practice management software for enhanced record-keeping. The system's advanced AI capabilities (FIG. 13) evaluate facial landmarks to generate automated smile simulations, providing patients with a visual representation of potential outcomes.

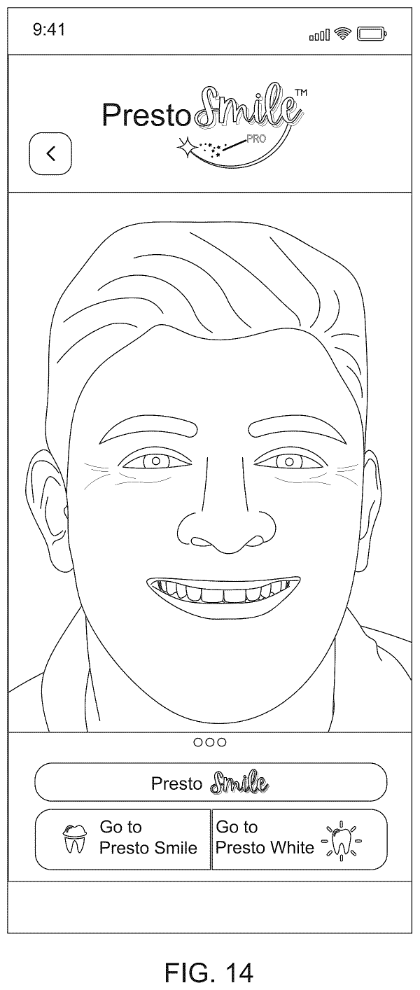

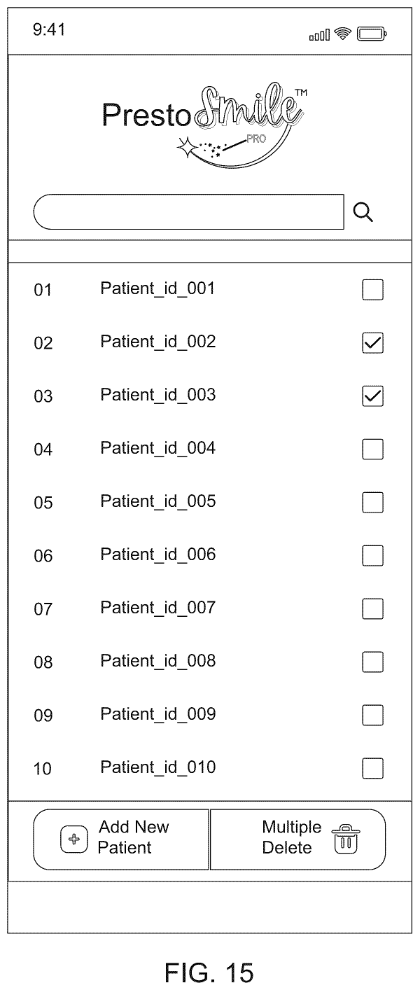

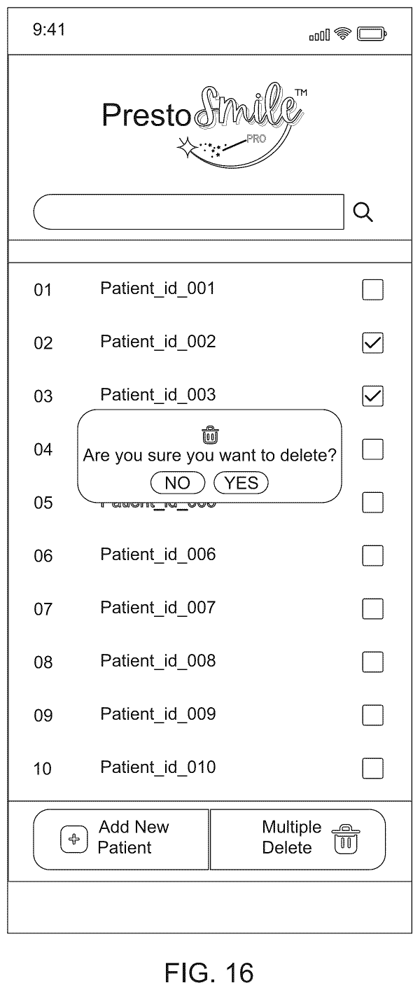

Furthermore, FIGS. 14 and 15 display the process of editing patient details and managing patient IDs within the system, allowing for efficient record-keeping and organization. Users can delete patient folders (FIG. 16) or view patient records in a folder view format for easy access and management.

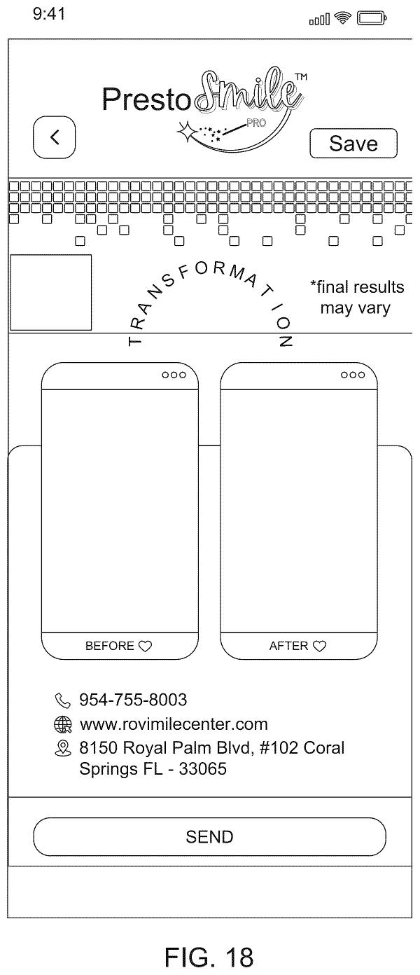

Lastly, FIG. 18 illustrates the transformation of smile possibilities before and after treatment, with a disclaimer stating that final results may vary. The system also includes detailed profile information for dentists and a dedicated help and support section for customer care inquiries, ensuring a seamless user experience and effective practice management.

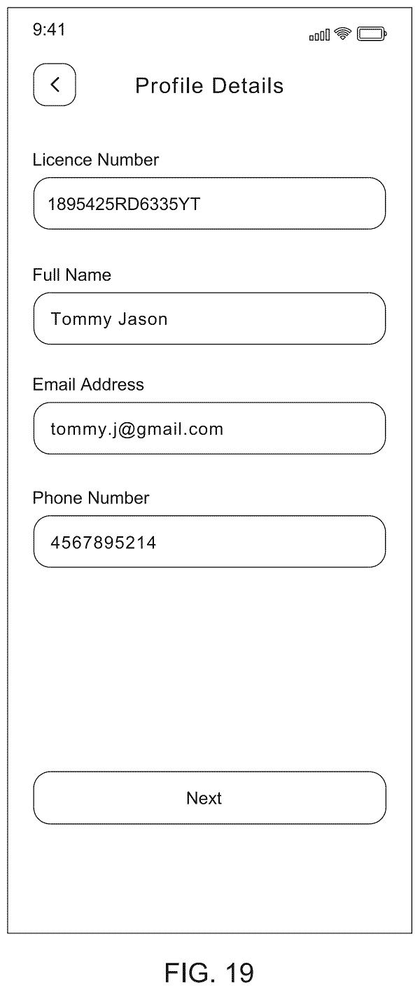

In FIG. 19, users can access detailed profile information for doctors, providing essential details about their qualifications, expertise, and professional background. This feature enables patients to make informed decisions when selecting a healthcare provider and fosters transparency within the practice.

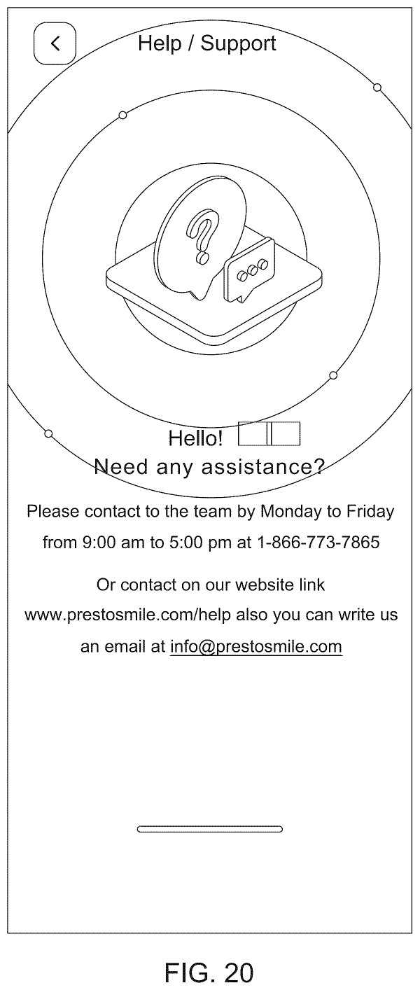

FIG. 20 showcases the help support page for customer care, offering users a centralized platform to seek assistance, report issues, or provide feedback. The system's customer support team is readily available to address inquiries, troubleshoot technical issues, and ensure a positive user experience for all stakeholders.

FIG. 21 highlights the advanced technology underpinning the dental imaging system, including state-of-the-art AI algorithms, image processing capabilities, and interactive video consultation tools. The system's innovative technology stack empowers dental practices to deliver personalized care, streamline administrative tasks, and enhance patient engagement through digital solutions.

By leveraging cutting-edge technology and AI-driven functionalities, Presto Smile sets a new standard for dental practice management systems, offering a comprehensive suite of features to meet the evolving needs of modern healthcare providers and patients alike. Presto Smile represents a groundbreaking AI-powered solution for dental practices, offering a comprehensive set of features to enhance patient interactions, streamline workflows, and improve overall practice efficiency.

The dental imaging system can also communicate with one or more external APIs. The one or more external APIs can include interfaces to external databases, social media platforms, search engines, file servers, web servers, or any type of computerized resource that can communicate with the dental imaging system.

The dental imaging system can also communicate with the administration system of dental providers, for example, via web browsers. The dental imaging system can include dentist schedulers 1-N for sending, receiving, processing, or any combination thereof prospective patient and dentist records data, such as profile data, calendar data, smile profile data, and edits or updates thereto. The dental imaging system can be configured to send, receive, create, process, or any combination thereof algorithmic models, datasets, tagged datasets, prospective patient records data, dentist records data, or any combination thereof.

The administrator devices 1-N of the dental practices can send, receive, create, process, or any combination thereof statistical algorithms, algorithmic models, datasets, dataset tags, federated rules, processes, standards, practices, or any combination thereof, and other system, e.g., Unix or Unix-like, administration functions, such as database administration, system access, authorization, and authentication, and security (PKI, e.g.). Records data can include profile data, calendar data, smile profile data, or edits and updates thereto. Federated, as used herein, refers to systems and methods that process, communicate, or both data using agreed upon data structures, information, information sources, methods of processing, methods of communications, practices of processing, practices of communications, or any combination thereof.

In the embodiments described above, the computer devices of the dental imaging system and/or the user devices can include a processing device coupled to a communication device. The processing device can be coupled to a memory device, and an input/output (“I/O”) interface. In embodiments, the communication interface enables the computer devices of the dental imaging system and/or the user devices to communicate with other devices and systems via one or more networks. The processing device, the communication device, the memory device, and the I/O interface can be interconnected via a system bus. The system bus can be and/or include a control bus, a data bus, an address bus, and the like. The processing device can be and/or include a processor, a microprocessor, a computer processing unit (“CPU”), a graphics processing unit (“GPU”), a neural processing unit, a physics processing unit, a digital signal processor, an image signal processor, a synergistic processing element, a field-programmable gate array (“FPGA”), a sound chip, a multi-core processor, and the like. As used herein, “processor,” “processing component,” “processing device,” and/or “processing unit” can be used generically to refer to any or all of the aforementioned specific devices, elements, and/or features of the processing device.

The memory device can be and/or include one or more computerized storage media capable of storing electronic data temporarily, semi-permanently, or permanently. The memory device can be or include a computer processing unit register, a cache memory, a magnetic disk, an optical disk, a solid-state drive, and the like. The memory device can be and/or include random access memory (“RAM”), read-only memory (“ROM”), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device.

The communication device enables the computer devices of the dental imaging system and/or the user devices to communicate with other devices and systems. The communication device can include hardware and/or software for generating and communicating signals over a direct and/or indirect network communication link. As used herein, a direct link can include a link between two devices where information is communicated from one device to the other without passing through an intermediary. For example, the direct link can include a Bluetooth™ connection, a Zigbee connection, a Wifi Direct™ connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network. programming installed on a processor, such as the processing component, coupled to the antenna.

An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

In embodiments, the components and functionality of the dental imaging system can be hosted and/or instantiated on a “cloud” and/or “cloud service.” As used herein, a “cloud” and/or “cloud service” can include a collection of computer resources that can be invoked to instantiate a virtual machine, application instance, process, data storage, or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

In embodiments, the components and functionality of the dental imaging system can be and/or include a “server” device. The term server can refer to functionality of a device and/or an application operating on a device. The server device can include a physical server, a virtual server, and/or cloud server. For example, the server device can include one or more bare-metal servers such as single-tenant servers or multiple-tenant servers. In another example, the server device can include a bare metal server partitioned into two or more virtual servers. The virtual servers can include separate operating systems and/or applications from each other. In yet another example, the server device can include a virtual server distributed on a cluster of networked physical servers. The virtual servers can include an operating system and/or one or more applications installed on the virtual server and distributed across the cluster of networked physical servers. In yet another example, the server device can include more than one virtual server distributed across a cluster of networked physical servers.

Various aspects of the systems described herein can be referred to as “content” and/or “data.” Content and/or data can be used to refer generically to modes of storing and/or conveying information. Accordingly, data can refer to textual entries in a table of a database. Content and/or data can refer to alphanumeric characters stored in a database. Content and/or data can refer to machine-readable code. Content and/or data can refer to images. Content and/or data can refer to audio and/or video. Content and/or data can refer to, more broadly, a sequence of one or more symbols. The symbols can be binary. Content and/or data can refer to a machine state that is computer-readable. Content and/or data can refer to human-readable text.

The computer devices of the dental imaging system and/or the user devices can include a user interface for outputting information in a format perceptible by a user and receiving input from the user, for example, the GUIs discussed above. The user interface can include a display screen such as a light-emitting diode (“LED”) display, an organic LED (“OLED”) display, an active-matrix OLED (“AMOLED”) display, a liquid crystal display (“LCD”), a thin-film transistor (“TFT”) LCD, a plasma display, a quantum dot (“QLED”) display, and so forth. The user interface can include an acoustic element such as a speaker, a microphone, and so forth. The user interface can include a button, a switch, a keyboard, a touch-sensitive surface, a touchscreen, a camera, a fingerprint scanner, and so forth. The touchscreen can include a resistive touchscreen, a capacitive touchscreen, and so forth.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the above is a complete description of specific examples of the disclosure, additional examples are also possible. Thus, the above description should not be taken as limiting the scope of the disclosure which is defined by the appended claims along with their full scope of equivalents.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

It should be understood, of course, that the foregoing relates to exemplary embodiments of the disclosure and that modifications can be made without departing from the spirit and scope of the disclosure as set forth in the following claims.

What is claimed is:

1. A method of generating a prop, comprising:
capturing at least one image of a face of a user;
performing preprocessing on at least one image;
receiving a selection of one smile template by the user;
applying the at least one image and the selection to one or more machine learning algorithms, wherein the one or machine learning algorithms are trained to generate one or more modified images that transform a smile based on the selection of one smile template and trained to output one or more procedures required to transform the smile; and
transmitting the one or more modified images and the one or more procedures to the user.
